(19) United States
(12) Patent Application Publication
(10) Pub. No.: US 2009/0240329 A1
(43) Pub. Date: Sep. 24, 2009

(54) Compounds Suitable as Polymerizable Yellow Dyes; Polymerizable and/or Crosslinkable Compositions, Polymer Matrices and Intraocular Lenses Containing them

(75) Inventor: Cecile Gousse, Dingy Saint Clair (FR)

Correspondence Address: COHEN, PONTANI, LIEBERMAN & PAVANE LLP, 551 FIFTH AVENUE, SUITE 1210, NEW YORK, NY, 10176, US

(73) Assignee: Corneal Innovation, Paris (FR)

(21) Appl. No.: 11/992,462

(22) PCT Filed: Sep. 27, 2006
(86) PCT No.: PCT/FR2006/050945

§ 371 Date: Dec. 29, 2008

(30) Foreign Application Priority Data
Sep. 28, 2005 (FR) 0509882

(51) Int. Cl. A61F 2/16; C09B 27/00; C09B 43/11; C08L 33/24

(52) U.S. Cl. 623/6.11; 534/850; 534/588; 525/218

(57) ABSTRACT

The present invention relates to:
novel compounds which are polymerizable functionalized derivatives of the yellow dye product known as C.I. Disperse Yellow 3, listed under CAS no. 2832-40-8, and have the formula

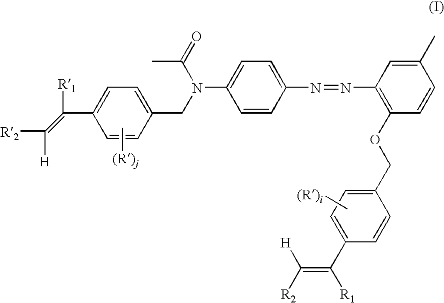

the preparation of said novel compounds;
their use as yellow dyes;
polymerizable and/or crosslinkable compositions containing them;
colored polymer matrices obtainable from such compositions; and finally
intraocular lenses consisting wholly or partly of such colored polymer matrices.

The present invention principally relates to compounds suitable as polymerizable yellow dyes and to their use for coloring more particularly intraocular lenses.

The present invention relates more precisely to:
novel compounds which are polymerizable functionalized derivatives of the yellow dye product known as C.I. Disperse Yellow 3, listed under CAS no. 2832-40-8 (i.e. 4-(2-hydroxy-5-methylphenylazo)acetanilide);
the preparation of said novel compounds;
their use as yellow dyes;
polymerizable and/or crosslinkable compositions containing them;
colored polymer matrices obtainable from such compositions; and finally
intraocular lenses consisting wholly or partly of such colored polymer matrices.

The present invention has been developed in the context of the surgical treatment of cataract and intraocular lenses.

Elderly people whose natural lens has been replaced with an intraocular lens generally suffer from cyanopsia: the objects around them tend to appear blue, which is the complementary color to yellow. This modification of vision is explained by the fact that the transmission spectrum of the natural lens changes with age due to a yellowing of said natural lens, whereas the intraocular lens that replaces said aged, i.e. yellowed, natural lens is colorless. It has thus been proposed for some years to correct this modification of vision by using yellow intraocular lenses.

Such yellow intraocular lenses have also been proposed in the context of the possible damage which blue light (400-500 nm) might cause to the retina.

A very large number of parameters are involved in the production of these colored intraocular lenses, as in that of colored contact lenses:
the nature of the yellow dye in question,
the nature of the matrix in which it is used,
the mode of use of said dye in said matrix: simple dispersion or true fixation (the purpose of any fixation is to avoid elimination of the dye by elution),
the type of true fixation in question: the nature of the chemical groups involved, the optional insertion of a spacer group in the chemical structure of the dye, etc.

The aim is quite obviously to provide stable high-performance products under advantageous conditions of preparation.

Products of this type have been described in the patent literature, especially by the following companies: Menikon, Hoya, Alcon and Canon-Staar.

Menikon has proposed the following:
in patent application JP-A-1 280 464: soluble dyes whose chemical formula contains a polymerizable group of the acryloyloxy, methacryloyloxy, vinyl or allyl type, and which are intended to be used as comonomers in the preparation of colored polymer matrices;
in patent application JP-A-2 232 056: polymerizable compounds which are both dyes and UV filters, and which are also intended for use in polymer matrices;
in patent application EP-A-634 518: a process for the production of tinted contact lenses;
in U.S. Pat. No. 6,242,551: polymerizable dyes which are also intended for use in polymer matrices.

Hoya has proposed the following:
in patent application EP-A-359 829: a process for the production of intraocular lenses which are capable of correcting cyanopsia and whose constituent material contains a yellow, yellow-brown or orange dye;
in patent application EP-A-799 864: polymerizable quinone dyes and the soft contact lenses containing them;
in patent application JP-A-10 195 324: a polymerizable yellow dye of the pyrazolone family and its use in the production of soft intraocular lenses by copolymerization;
in patent application EP-A-1 043 365: polymerizable 1,2-dipyrazoethenes which are also suitable as polymerizable yellow dyes for plastic ophthalmic lenses.

Alcon has proposed the following:
in patent application EP-A-674 684: other polymerizable yellow dyes (obtained by a two-step synthesis) and their use for the production of ophthalmic lenses. Said dyes are polymerizable by virtue of the presence, in their formula, of at least one polymerizable group selected from acrylic and methacrylic groups. According to the teaching of said document, these acrylic-type groups have been shown to perform better than vinylic-type groups.

Finally, Canon-Staar has proposed the following:
in patent application US-A-2003/0078359: two novel families of polymerizable dyes, the incorporation of such dyes into silicone material, and intraocular lenses containing said dyes.

In such a context, the Applicant now proposes a novel family of polymerizable yellow dyes.

Said dyes, which are bifunctional, are particularly valuable as polymerizable products by virtue of their ease of preparation, their stability and their reactivity, and as dyes for coloring intraocular lenses by virtue of their color, which is capable of imparting to said lenses a UV-visible spectrum similar to that of the natural lens of a 25-year-old man. In fact, said dyes are capable of imparting:
a quasi-zero transmission up to 400 nm,
a constant increase in transmission between 400 and 500 nm, and then
a maximum transmission at the higher wavelengths (corresponding to non-harmful rays).

According to its first subject, the present invention therefore relates to novel compounds of formula (I) below:

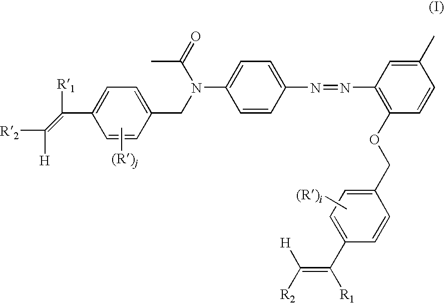

in which
R and R′ independently are a linear alkyl or hydroxyalkyl group containing from 1 to 4 carbon atoms;
i and j independently are an integer selected from 0, 1, 2, 3 and 4, it being possible for the different R, when i=2, 3 or 4, to be identical or different, and it being possible for the different R′, when j=2, 3 or 4, to be identical or different; and
R₁, R₂, R′₁ and R′₂ independently are hydrogen or a methyl group.

Said novel compounds constitute the novel dyes referred to above.

In formula (I) above, when i has other than the value zero, R is advantageously linear alkyl or hydroxyalkyl containing 1 or 2 carbon atoms; when j has other than the value zero, R′ is advantageously linear alkyl or hydroxyalkyl containing 1 or 2 carbon atoms.

In said formula (I) above, the following definitions are advantageous:

(R′)ⱼ=(R)ᵢ,

R′₁=R₁, and

R′₂=R₂.

In said formula (I) above, the following definitions are very advantageous: i=j=0 and R₁=R₂=R′₁=R′₂=H.

The compound of formula (Ia) below:

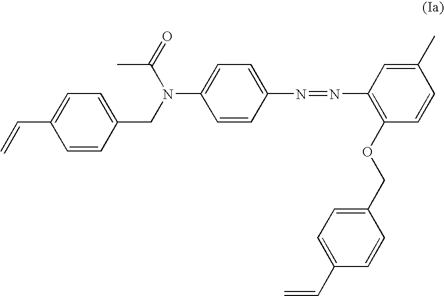

is thus particularly preferred.

According to its second subject, the present invention relates to the process for the preparation of the novel compounds of formula (I) above. It is an analogy process, i.e. a process of etherification of the hydroxyl group and alkylation of the amide group of the compound of formula (II) below (4-(2-hydroxy-5-methyl-phenylazo)acetanilide):

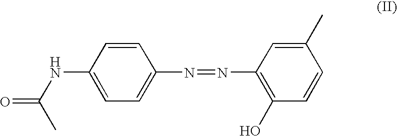

Said etherification and alkylation process is carried out in an alkaline, polar, aprotic solvent medium in the presence of a strong base such as KOH. It is advantageously carried out in dimethyl sulfoxide (DMSO) or dimethylformamide (DMF).

In said process, said compound of formula (II) above is reacted with at least one styrene derivative of formula (III):

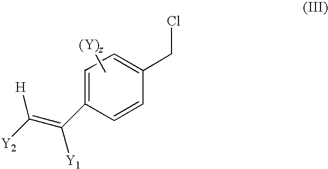

in which
Y is a linear alkyl or hydroxyalkyl group containing from 1 to 4 carbon atoms;
z is an integer selected from 0, 1, 2, 3 and 4, it being possible for the different Y, when z=2, 3 or 4, to be identical or different; and
Y₁ and Y₂ independently are hydrogen or a methyl group,
said at least one styrene derivative being used in stoichiometric excess.

When one compound of formula (III) is used, the compound of formula (I) obtained has two similar ends.

To prepare the preferred compound of formula (Ia), the hydroxyl and amide groups of the compound of formula (II) are respectively etherified and alkylated with 4-chloromethylstyrene (unique compound of formula III to be used).

When at least two different compounds of formula (III) are used, a mixture of several compounds of formula (I) is obtained, namely compounds of formula (I) having two similar ends and compounds of formula (I) having different ends. Compounds of a given formula (I) can be isolated from such mixtures in a manner known per se.

The styrene derivative of formula III (or the styrene derivatives of formula III, taken as a whole) is (are) always used in stoichiometric excess.

The aim is thus to react the hydroxyl and amide groups of the compound of formula (II).

In general, said at least one styrene derivative is used in an amount corresponding to at least twice the stoichiometric amount and advantageously in an amount corresponding to at least three times the stoichiometric amount.

The preparation of the compounds of formula (III)—styrene derivatives—does not present those skilled in the art with any particular difficulties.

As indicated above, the compound of formula (II), listed under CAS no. 2832-40-8, is known as C.I. Disperse Yellow 3. It is a high-performance yellow dye.

Starting from such a known compound (known dye), the process of the invention makes it possible in a single step (constituting a definite advantage compared with the process according to the teaching of EP-A-674 684) to obtain the novel compounds of formula (I) of the invention. These are particularly valuable compounds which themselves are high-performance dyes.

According to its third subject, the present invention relates to such dyes, in other words to the use of the compounds of formula (I) as dyes. These novel dyes are particularly valuable and high-performance for the following reasons:
their synthesis comprises only one step;
the ether and amide groups which carry the polymerizable groups are not cleavable under physiological conditions;
the polymerizable group—styrene group—is very reactive and generates very stable bonds after reaction;
said novel dyes have virtually retained the color of the compound from which they are derived, namely C.I. Disperse Yellow 3 (in another connection, a small color change is observed between the polymerizable and/or crosslinkable compositions that contain the dye in disperse form and the polymer matrices that contain said dye in covalently bonded form, the former generally being yellow-brown and the latter generally being yellow-orange);
said dyes make it possible to prepare matrices whose UV-visible spectra are similar to that of a 25-year-old man.

The other subjects of the invention all relate to the use of the novel compounds of formula (I) as dyes in:
polymerizable and/or crosslinkable compositions, and
colored polymer matrices obtainable from such compositions and useful especially as the constituent material of intraocular lenses.

Thus, according to its fourth subject, the present invention relates to compositions which are polymerizable and/or crosslinkable by a free-radical process and comprise a mixture of:
precursors of a polymer matrix; and
an effective amount of at least one dye of the invention (a compound of formula (I)).

Said composition can be any composition that is convertible to a matrix in which the dye(s) has (have) been trapped and fixed via at least one of its (their) reactive polymerizable groups.

The precursors in question can be monomers and the dyes of the invention constitute comonomers in the mixtures. This type of mixture is copolymerized and generally crosslinked as well in order to generate a coherent polymer matrix.

The precursors in question can be polymers, in which case the dyes of the invention are fixed to their skeleton beforehand and/or when they are crosslinked.

Irrespective of the exact contexts in which the dyes of the invention are used, they are quite obviously used in an effective amount for imparting the desired color to the final product, imparting the desired color to the composition that is the precursor of said final product. The effective amount in question is generally between 0.02 and 0.15% by weight of the mixture (mainly: precursors of the matrix+dye(s), i.e. the reaction mixture).

The mixtures in question, which are destined to react in order to generate a matrix in which the dye is fixed, generally contain the following in conventional manner:

an effective amount of at least one polymerization initiator; and/or

an effective amount of at least one crosslinking agent.

Reagents of this type are used very particularly in a mixture of comonomers to be copolymerized.

As an initiator of the free-radical copolymerization, by a thermal process, particularly of comonomers of acrylic type, it is recommended to use:
a mixture of sodium phosphite and sodium phosphate (or any other redox couple);
   an azo compound such as azobisisobutyronitrile (AIBN) or 2,2′-azobis-(2,4-dimethylvaleronitrile) (AIVN), which is marketed in particular by WAKO under the reference V65, the structural formulae of these compounds being as shown below:

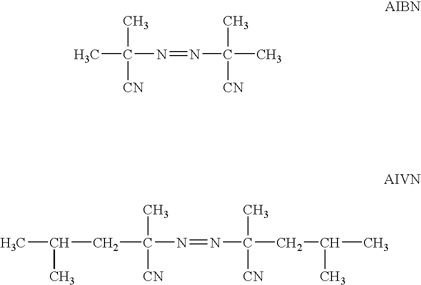

the latter compound being particularly preferred for its low toxicity, as well as that of its degradation products (it is noted, however, that in general the polymerization initiator is used in a very small amount and is generally removed at the end of the process for the production of the intraocular lenses of the invention); or
a peroxide such as benzoyl peroxide.

Those skilled in the art know how to control the amount of said free-radical polymerization initiator used (generally less than 1 part by weight per 100 parts by weight of monomers to be copolymerized) and, in general, the polymerization kinetics of the reaction medium. They know in particular that, as oxygen neutralizes the action of said polymerization initiator, it is highly preferable to remove it from the reaction mixture before the temperature rises. It is highly recommended to bubble inert gas through said reaction mixture. As far as the heating program is concerned, its optimization is within the capability of those skilled in the art.

The preparation of compositions polymerizable by a photochemical process is not excluded within the framework of the invention. In this case an effective amount of at least one conventional photoinitiator, e.g. of benzophenone type, is used in said compositions.

Reference has also been made to an effective amount of at least one crosslinking agent. Such crosslinking can prove essential for ensuring the cohesion of the matrix and its stability. A crosslinking agent (at least bifunctional) is therefore generally used, in an effective amount, during the copolymerization of the comonomers or the crosslinking of the polymers. This effective amount—generally at most a few parts by weight: in principle between 0.5 and 5 parts by weight, advantageously between 0.5 and 2.5 parts by weight, per 100 parts by weight of reactants—must obviously remain reasonable. For example, the crosslinking agent used should not constitute a “comonomer” and consequently modify the properties, especially mechanical properties, of the expected copolymer.

Whatever the case may be, those skilled in the art are not unaware that increasing the proportion of crosslinking agent used reduces the water content of the hydrogels and increases their glass transition temperature.

As regards the reactive groups of said crosslinking agent, they are advantageously acrylate and/or methacrylate groups. Those skilled in the art are familiar with numerous crosslinking agents that carry such groups, especially the following:

butanediol dimethacrylate and diacrylate,

hexanediol dimethacrylate and diacrylate,

decanediol dimethacrylate and diacrylate,

ethylene glycol dimethacrylate (EDMA),

tetraethylene glycol dimethacrylate.

Within the framework of the present invention, it is recommended, without in any way implying a limitation, to use the crosslinking agents listed above, and very particularly EDMA.

Thus the polymerizable and/or crosslinkable compositions of the invention generally contain crosslinking agents of this type (or of an equivalent type), traces of which are obviously found in the polymer matrices obtained from said compositions.

In the context of producing intraocular lenses, those skilled in the art understand that said compositions may also generally contain an effective amount of at least one ultraviolet absorber. Those skilled in the art do not ignore the interest for a compound of this type, which will act as a UV filter, to be stabilized in the structure of intraocular lenses. The sought-after stabilization will be optimal if said ultraviolet absorber is itself also copolymerizable, i.e. has an appropriate reactive chemical entity, such as a double bond or an acrylate or methacrylate group, in its formula.

Those skilled in the art are familiar with such compounds and several of them are commercially available, especially:
4-(2-acryloxyethoxy)-2-hydroxybenzophenone,
4-methacryloxy-2-hydroxybenzophenone (MOBP),
1,3-bis(4-benzoyl-3-hydroxyphenoxy)-2-propyl acrylate,
2-(2′-methacryloxy-5′-methylphenyl)benzotriazole.

All these compounds are suitable within the framework of the present invention, but the Applicant has more particularly used MOBP insofar as it has been handling this UV filter for many years in the manufacture of intraocular lenses.

The UV filters are generally used in an amount of 0.25 to 5 parts by weight, advantageously of 0.25 to 2 parts by weight, in the reactive mixture.

The polymerizable and/or crosslinkable compositions of the invention are based on polymer matrix precursors containing an effective amount of at least one dye of the invention.

Advantageously, said polymer matrix precursors are selected from polysiloxanes and monomers of acrylic type. Very advantageously, they are selected from monomers of acrylic type.

The (crosslinkable) polysiloxanes used are known to those skilled in the art. There are actually two solutions to be handled:

the first containing at least one polysiloxane with vinyl end groups and a catalyst,

the second containing at least one polysiloxane with vinyl end groups and a polysiloxane with silicon hydride groups.

When these two solutions are brought into contact, the groups

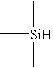

react with the groups

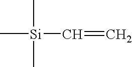

to form a resin (matrix). When dye of the invention is present, it becomes fixed and trapped within said resin.

The monomers of acrylic type in question can fall into two categories: they can be suitable for generating a hydrophilic matrix or for generating a hydrophobic matrix. Conventionally, hydrophilic refers to a matrix with hydroxyl groups that allow said matrix to absorb water. Such a matrix is also called a hydrogel.

In the context of hydrophilic matrices, preferred precursor mixtures are those based on:
2-hydroxyethyl methacrylate (HEMA) and
at least one C1-C8 alkyl or hydroxyalkyl (meth)acrylate.

Mixtures of the following types are very particularly preferred:
2-hydroxyethyl methacrylate (HEMA) and methyl methacrylate (MMA), especially the mixtures from which MENTOR produced lenses marketed at the time under the name MEMORYLENS®;
2-hydroxyethyl methacrylate (HEMA) and ethyl methacrylate (EMA), especially the mixtures described in FR-A-2 757 065; or
2-hydroxyethyl methacrylate (HEMA) and 6-hydroxyhexyl methacrylate (HHMA), especially the mixtures described in U.S. Pat. No. 5,217,491.

In the context of hydrophobic matrices, preferred precursor mixtures are those based on monomers of the formula

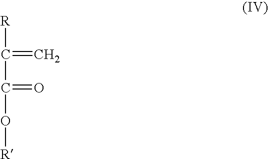

in which R is hydrogen or a methyl group and R′ is a linear or branched alkyl group containing from 1 to 5 carbon atoms or a (C1-C5 alkyl)phenyl group.

Very particularly preferred mixtures are those based on methyl methacrylate (MMA) or those based on ethyl acrylate (EA) and ethyl methacrylate (EMA) (especially the mixtures described in EP-A-674 684).

According to its fifth subject, the present invention relates to the colored polymer matrices obtainable by free-radical copolymerization and/or crosslinking of the polymerizable and/or crosslinkable compositions described above. Said matrices characteristically contain the yellow dye of formula (I), said yellow dye being stably contained therein. It forms an integral part of said matrices. It has reacted via its double bond(s) in order to enter their structure.

The colored matrices are advantageously of polysiloxane or acrylic type (cf. above).

According to its last subject, the present invention finally relates to intraocular lenses consisting wholly or partly of colored polymer matrices of the invention. At least the optics of said lenses are thus made up of colored polymer matrices of the invention. Advantageously, the optics and the haptics of said lenses are made up of colored polymer matrices of the invention.

Such lenses are particularly high-performance in view of the advantages stated above in the present text for the compounds of formula (I) used as a yellow dye within them.

The invention is illustrated by the following Examples and the appended Figures.

The first step was to prepare a polymerizable yellow dye according to the invention—the compound of formula (Ia)—and this was then incorporated into polymerizable and crosslinkable compositions, which were polymerized and crosslinked in order to generate colored polymer matrices that were perfectly suitable as a constituent material of intraocular lenses.

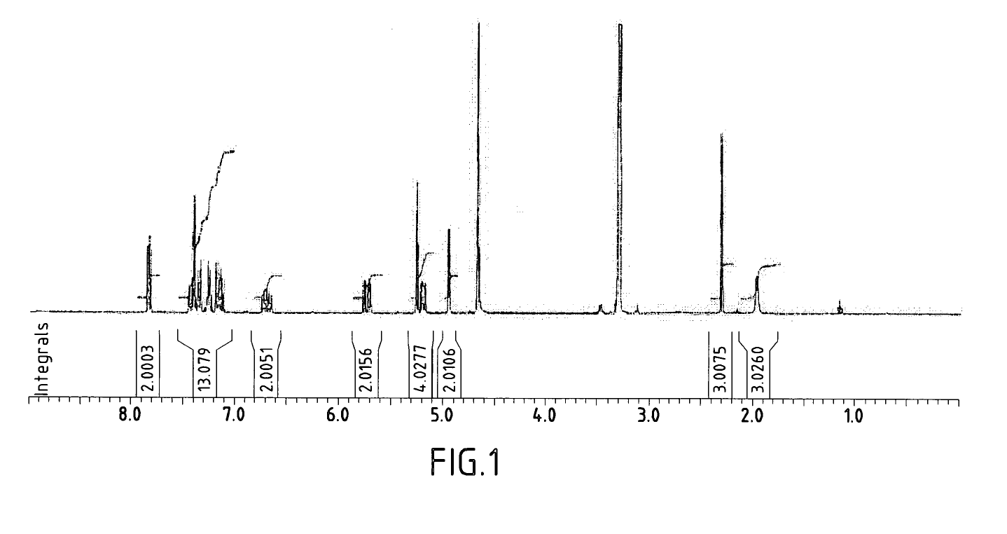

FIG. 1 shows the ¹H NMR spectrum of said compound of formula (Ia), as prepared. The following characteristic peaks are found in said spectrum:

aromatic rings between 7 and 8 ppm,

vinylic bonds at 6.70, 5.75 and 5.20 ppm,

CH₂—N at 5.25 ppm,

CH₂—Ar at 4.95 ppm,

CH₃—Ar at 2.31 ppm and

CH₃—CO—N at 1.97 ppm.

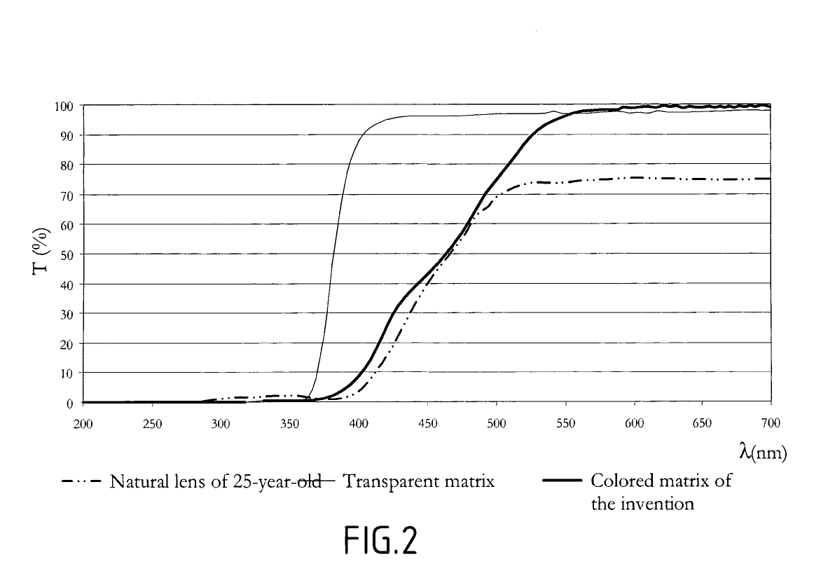

Transmission spectra are shown in FIG. 2, which is discussed below.

### EXAMPLE

### Preparation of the Compound of Formula (Ia)

Said preparation is carried out at room temperature.

1 g of C.I. Disperse Yellow 3 (CAS no. 2832-40-8) is introduced into a round-bottomed flask and then covered with 50 ml of dimethylformamide. The whole is stirred for 1 h. The addition of 0.32 g of potassium hydroxide causes the solution to turn violet. Stirring is then maintained for 40 min.

4-Chloromethylstyrene (0.48 ml) is then added. The reaction medium is stirred for 42 h.

When these 42 h have elapsed, 100 ml of deionized water are added to said reaction medium.

The monomer (compound of formula (Ia)) is extracted with ether. The organic phase is subsequently washed with 5 times 100 ml of deionized water and then dried over sodium sulfate; the solvent is evaporated off.

The monomer is recrystallized from ether previously dried over sodium sulfate.

### Preparation of Hydrophilic Colored Matrices

The compound of formula (Ia) is weighed into a glass flask and then added to a solution of monomers containing:
82.5 g of 2-hydroxyethyl methacrylate (HEMA),
17.5 g of ethyl methacrylate (EMA),
0.8 g of ethylene glycol dimethacrylate and
0.5 g of 4-methacryloxy-2-hydroxybenzophenone,
   in such a way that the concentration of dye is between 0.02 and 0.15% by weight, depending on the sample.

Benzoyl peroxide (0.2 g) is then added.

The mixture is homogenized by ultrasound and degassed with argon. Polymerization (crosslinking) is then effected at 40° C. for 96 h. Subsequent curing of the blanks is assured by a thermal post-polymerization phase of 3 hours at 100° C.

The efficacy of the polymerization (crosslinking) reaction, or, more precisely, that of the trapping of the dye, was evaluated in the manner specified below.

Hydrated pellets were extracted for a minimum of 6 hours with a mixture of equal volumes of methanol and distilled water. The UV-visible spectra of said pellets before and after extraction were compared.

The loss of absorbance of said pellets remained below 10% between 400 and 500 nm and reached 20% for the weak transmissions between 350 and 400 nm. Such a loss of absorbance is not a serious problem.

Furthermore, the material obtained offers a good filtering power for the blue rays which is similar to that obtained by the natural lens of a 25-year-old man, and a very good transmission for the wavelengths beyond the blue.

### Preparation of Other Hydrophilic Colored Matrices

The compound of formula (Ia) is weighed into a glass flask and then added to a solution of monomers containing:
82.5 g of 2-hydroxyethyl methacrylate (HEMA),
17.5 g of methyl methacrylate (MMA),
0.8 g of ethylene glycol dimethacrylate and
0.5 g of 4-methacryloxy-2-hydroxybenzophenone,
   in such a way that the concentration of dye is between 0.02 and 0.15% by weight, depending on the sample.

Benzoyl peroxide (0.2 g) is then added.

The mixture is homogenized by ultrasound and degassed with argon. Polymerization (crosslinking) is then effected at 40° C. for 96 h. Subsequent curing of the blanks is assured by a thermal post-polymerization phase of 3 hours at 100° C.

The material obtained offers a good filtering power for the blue rays which is similar to that obtained by the natural lens of a 25-year-old man, and a very good transmission for the wavelengths beyond the blue. This is shown in appended FIG. 2.

Said FIG. 2 shows three transmission spectra, T=f(λ):
the first (- . . . -) representing the natural lens of a 25-year-old man,
the third (-) representing a colored matrix (HEMA+MMA+dye of the invention in a proportion of 0.075% by weight), as prepared above,
and the second (-) representing the corresponding transparent matrix (HEMA+MMA), prepared in the same manner without the use of dye.

### Preparation of Hydrophobic Colored Polymer Matrices

The compound of formula (Ia) is weighed into a glass flask and then added to a solution of monomers containing:
100 g of methyl methacrylate (MMA),
0.8 g of ethylene glycol dimethacrylate and
0.5 g of 4-methacryloxy-2-hydroxybenzophenone,
   in such a way that the concentration of dye is between 0.02 and 0.15% by weight, depending on the sample.

Benzoyl peroxide (0.2 g) is then added.

The mixture is homogenized by ultrasound and degassed with argon. Polymerization (crosslinking) is then effected at 40° C. for 96 h. Subsequent curing of the blanks is assured by a thermal post-polymerization phase of 3 hours at 100° C.

The material obtained offers a good filtering power for the blue rays which is similar to that obtained by the natural lens of a 25-year-old man, and a very good transmission for the wavelengths beyond the blue.

1. Compounds of the formula 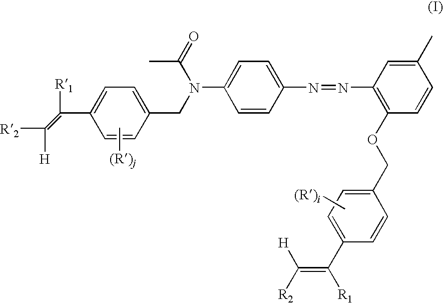
in which
R and R′ independently are a linear alkyl or hydroxyalkyl group containing from 1 to 4 carbon atoms;
i and j independently are an integer selected from 0, 1, 2, 3 and 4, it being possible for the different R, when i=2, 3 or 4, to be identical or different, and it being possible for the different R′, when j=2, 3 or 4, to be identical or different; and
R₁, R₂, R′₁ and R′₂ independently are hydrogen or a methyl group.

2. Compounds according to claim 1 of formula (I) in which
(R′)ⱼ=(R)ᵢ,
R′₁=R₁, and
R′₂=R₂.

3. Compound according to claim 1 of the formula

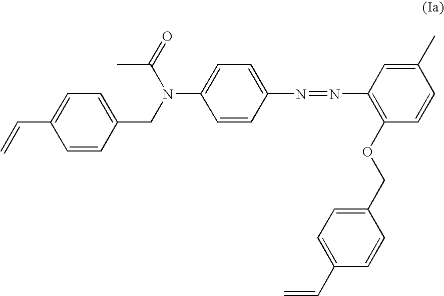

4. Process for the preparation of a compound according to claim 1, wherein the process comprises reacting the compound of formula (II): 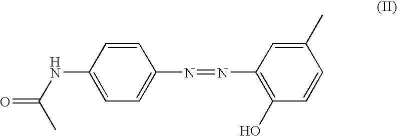
in an alkaline, polar, aprotic solvent medium, with at least one styrene derivative of formula (III): 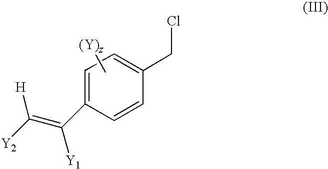
in which
Y is a linear alkyl or hydroxyalkyl group containing from 1 to 4 carbon atoms;
z is an integer selected from 0, 1, 2, 3 and 4, it being possible for the different Y, when z=2, 3 or 4, to be identical or different; and
Y₁ and Y₂ independently are hydrogen or a methyl group,
said at least one styrene derivative being used in stoichiometric excess.

5. Process according to claim 4, wherein said at least one compound of formula (III) is used in an amount corresponding to at least twice the stoichiometric amount and advantageously at least three times said stoichiometric amount.

6. Process according to claim 4, wherein the process comprises reacting said compound of formula (II) with 4-chloromethylstyrene.

7. Polymerizable yellow dye consisting of a compound according to claim 1.

8. Composition which is polymerizable and/or crosslinkable by a free-radical process, wherein the composition comprises a mixture of:
precursors of a polymer matrix; and
an effective amount of at least one polymerizable yellow dye according to claim 7, said effective amount generally representing 0.02 to 0.15% by weight of said mixture.

9. Composition according to claim 8, wherein said mixture also contains an effective amount of at least one polymerization initiator and/or an effective amount of at least one crosslinking agent.

10. Composition according to claim 8, wherein said mixture also contains an effective amount of at least one ultraviolet absorber that is advantageously copolymerizable.

11. Composition according to claim 8, wherein said polymer matrix precursors are selected from polysiloxanes and monomers of acrylic type.

12. Composition according to claim 8, wherein said precursors are monomers of acrylic type that are suitable for generating a hydrophilic matrix.

13. Composition according to claim 12, wherein the monomers of acrylic type consist of 2-hydroxyethyl methacrylate (HEMA) and at least one C1-C8 alkyl or hydroxyalkyl (meth)acrylate.

14. Composition according to claim 12, wherein said monomers of acrylic type consist of the following mixtures:
2-hydroxyethyl methacrylate (HEMA) and methyl methacrylate (MMA);
2-hydroxyethyl methacrylate (HEMA) and ethyl methacrylate (EMA); or
2-hydroxyethyl methacrylate (HEMA) and 6-hydroxyhexyl methacrylate (HHMA).

15. Composition according to claim 8, wherein said precursors are monomers of acrylic type that are suitable for generating a hydrophobic matrix.

16. Composition according to claim 15, wherein said monomers of acrylic type have the formula 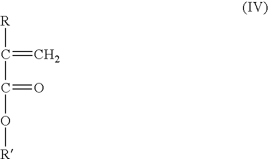
in which R is hydrogen or a methyl group and R′ is a linear or branched alkyl group containing from 1 to 5 carbon atoms or a (C1-C5 alkyl)phenyl group.

17. Colored polymer matrix obtainable by free-radical copolymerization and/or crosslinking of a polymerizable and/or crosslinkable composition according to claim 8.

18. Intraocular lenses possessing optics and haptics, wherein said optics (said optics and said haptics) are made up of a colored polymer matrix according to claim 17.
